(19) United States
(12) Patent Application Publication
(10) Pub. No.: US 2011/0230367 A1
(43) Pub. Date: Sep. 22, 2011

(54) Amphiphilic Peptides Promoting Production of Target miRNA and Method of Regulating Production of Target miRNA

(75) Inventors: Jaehoon Yu, Seoul (KR); Vic Narry Kim, Seoul (KR); Soonsil Hyun, Jeju-do (KR)

(73) Assignee: SNU R&DB FOUNDATION, Seoul (KR)

(21) Appl. No.: 13/122,055

(22) PCT Filed: Nov. 23, 2009
(86) PCT No.: PCT/KR09/06893

§ 371 Date: Mar. 31, 2011

(30) Foreign Application Priority Data
Oct. 22, 2009 (KR) 10-2009-0100572

(51) Int. Cl. C40B 30/04; C40B 30/06; C07K 2/00

(52) U.S. Cl. 506/9; 506/10; 530/300

(57) ABSTRACT

The present invention relates to an amphiphilic peptide capable of promoting target miRNA production and a method for regulating the production of target miRNA using the same. In detail, the amphiphilic peptide of the present invention binds strongly and specifically to hairpin-shaped target miRNA. The specific binding affinity induces the Dicer enzyme activity, therefore specifically increase the production of target miRNA. The present invention can be effectively used for regulating the amount of target miRNA produced in vivo, for the study of miRNA functions and for producing therapeutic drug for target miRNA related disease.

### BACKGROUND OF THE INVENTION

1. Field of the Invention

The present invention relates to amphiphilic peptides promoting production of a target miRNA and to a method of regulating the production of a target miRNA by using the same.

2. Description of the Related Art

The RNA that translates codes from DNA into protein is called a messenger RNA (mRNA), which consists only 2% of the total RNA. The rest of RNAs (98%) are non-coding RNAs. The non protein translating RNAs were considered insignificant and their functions were mostly unknown. However, there have been reports on the important functions of this non-coding RNA, by forming a complex with a gene or a protein, or functioning as an enzyme called ribozyme. Recently, there were reports on the regulatory function of these non-coding RNAs through specific interaction with the gene or the gene product. Therefore, it is becoming more important to understand the various functions and characteristics of these non-coding RNAs. The low molecular weight non-coding RNAs are called endogeneous microRNA (miRNA) or riboswitch, which controls more than 2% of the total gene regulatory function.

The most well known non-coding RNA is the microRNA (miRNA), and there are about 700 different types of miRNA in humans. Then miRNA is a single stand RNA molecule, consisting 19˜25 nucleotides. The miRNA is generated from an endogeneous hairpin-shaped transcript (Bartel, D. P., Cell 116:281-297, 2004; Kim, V. N., Mol. Cells. 19:1-15, 2005). The miRNA binds complementarily to a target mRNA and function as a post-transcriptional gene suppressor, which leads to a suppression of translation and also destabilizes the mRNA. There are also reports on a possible correlation between miRNA and cancer. In some reports, some of the miRNA have been shown to be related in development of cancer or its metastasis. Therefore, investigating the function of miRNA is being considered as a new and important area in cancer biology (Esquela-kerscher, A et al., Nat. Rev. Cancer 6(4):259-269, 2006). In addition, the expression level of miRNA dramatically changes during development and cell differentiation. The importance of miRNA in developmental system and in disease state has been confirmed by miRNA profiling. (Lu, J. et al., Nature 435:834-838, 2005). Reports have shown that one type of miRNA, let7a-1, is related in lung cancer or colon cancer, miR16-1 in leukemia or prostate cancer, miR24-1 in leukemia and let7a-1 in inhibiting general cancer promoting gene factors through inhibiting the Ras gene. In the case of miR16-1, it is capable of suppressing the grown of cancer cells by inhibiting the oncogenic factor, Bcl2. Therefore, discovering a method for and a reagent capable of regulating the amount of miRNA could be a candidate for an effective target therapeutic drug. In summary, we can increase the disease suppression by inhibiting the miRNA which causes the target disease and by activating the miRNA which has a function of suppressing the disease.

There are two enzymes that are involved in miRNA production in vivo. One is called Drosha, an endonuclease that is present in nucleus. The second is Dicer, an endonuclease that is present in cytosols. These two enzymes are RNA hydrolyzing enzymes, without base sequence specificity. They carry limited specificity through recognizing the length of the double stranded RNA for digesting. The primary miRNA (pri-miRNA) is formed into pre-miRNA of about 70˜90 nucleotides long stem-loop structure by RNaseIII type Drosha enzyme present in the nucleus. Next, the pre-miRNA is translocated to cytosol then digested by Dicer into a shorter 21˜25 nucleotide miRNA, called mature miRNA.

Since the production of miRNA is regulated by these two enzymes, understanding the specificity of these enzymes can be an important target for miRNA production. If the target miRNA cause disease such as cancer, these enzymes could be a good target protein for amplifying or reducing the disease causing target miRNA. In particular, the miRNA processing Dicer in the cytosol is considered as an important target since it can interact directly with the target mRNA in the cytosol.

However, a problem with the Dicer is that the processing of the enzyme is nonspecific and nonselective to the base sequence of pre-miRNA. Another miRNA processing enzyme, Drosha has a chaperon protein which binds to the substrate, therefore helping the nonspecific substrate become more specific. However, Dicer has the specificity of recognizing the length of the stem of the hairpin-shaped substrate and lacks the ability to specifically discriminate more than 700 other hairpin-shaped endogenous substrates. In vivo, a regulatory factor specifically maturing the miRNA is critical, but no such factor has been reported up till today.

Thus, the present inventors have conducted extensive research on a ligand which specifically binds to the target pre-miRNA and help produce a mature miRNA through adding artificial elements to induce specificity to the Dicer, which an enzyme unspecific to pre-miRNA base sequences. During this investigation, the present inventors have proved that the amphiphilic peptide constructed by substituting Tryptophan having indole group at the hydrophobic region forms a tight and specific interaction with the target pre-miRNA. This specific interaction promotes the Dicer enzyme activity, and specifically increases the production of mature miRNA, thereby leading to completion of the present invention.

### DETAILED DESCRIPTION OF THIS INVENTION

One object of the present invention is to provide amphiphilic peptides promoting production of target miRNA and to a method of regulating the production of target miRNA.

In order to achieve the object, the present invention provides an amphiphilic peptide library including one or more amphiphilic alpha helical peptide, wherein the alpha helical peptide has 4 to 12 Leu (leucine, L) at one, and of the hydrophobic amino acids, one or more Leu residues are substituted by Try (Trptophan, W).

The present invention also provides a method for detecting an amphiphilic peptide that binds specifically to a hairpin-shaped target pre-miRNA, including:
1) preparing the amphiphilic peptide library;
2) synthesizing a hairpin-shaped target pre-miRNA and one or more hairpin-shaped non target pre-miRNAs as a control;
3) measuring a binding affinity of the amphiphilic peptide for the pre-miRNA after combining the amphiphilic peptide, the hairpin-shaped target or non target pre-miRNA, and a probe molecule; and
4) selecting an amphiphilic peptide having a stronger binding affinity for target pre-miRNA than the non target pre-miRNA.

The present invention also provides a method for detecting a pre-miRNA which specifically binds to an amphiphilic peptide, including:
1) preparing the amphiphilic peptide library;
2) synthesizing a hairpin-shaped target pre-miRNA to be detected and one or more hairpin-shaped non target pre-miRNAs as a control to screen the amphiphilic library;
3) measuring a binding affinity of pre-miRNA for amphiphilic peptide after combining the amphiphilic peptide, the hairpin-shaped target or non target pre-miRNA, and a probe molecule; and
4) selecting the target pre-miRNA having stronger binding affinity to the amphiphilic peptide than the non target pre-miRNA.

The present invention also provides a method for detecting an amphiphilic peptide which increases a production of a target miRNA, including:
1) preparing the amphiphilic peptide library;
2) culturing a cell line after being treated with the amphiphilic peptide;
3) measuring the expression level of a target miRNA from the cell line; and
4) selecting an amphiphilic peptide which increases an expression level of the target miRNA compared to the non-treated control group.

The present invention also provides a composition for promoting a production of an amphiphilic peptide specific target miRNA selected by the above method, including the amphiphilic peptide as an active compound.

The present invention also provides a method for promoting a production of an amphiphilic peptide specific target miRNA selected by the above method, including administrating the amphiphilic peptide to a subject.

The present invention also provides a therapeutic drug or diagnostic reagent for disease caused by an inhibition of an amphiphilic peptide specific target miRNA selected by the above method, including the amphiphilic peptide as an active compound.

The present invention also provides a use of an amphiphilic peptide selected by the above method for preparing a composition for promoting a production of an amphiphilic peptide specific target miRNA.

The present invention also provides a use of an amphiphilic peptide selected by the above method for preparing a therapeutic drug or diagnostic reagent for disease caused by an inhibition of an amphiphilic peptide specific target miRNA.

Additionally, the present invention provides a method for promoting in vitro target pre-miRNA processing, including:
1) treating an amphiphilic peptide selected by the above method with pre-miRNA present in in vitro; and
2) binding the amphiphilic peptide and the target pre-miRNA.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a view showing the predicted secondary structure of two types of pre-miRNA pre-let7a-1(a) and pre-miR16-1(b) by using M-fold.

FIG. 2 is a graph showing the standardized initial reaction rate (Vₒ) of Dicer converting the pre-miRNA into mature miRNA in the presence of peptide 2b, 2c or 1e.

FIG. 3 is a graph showing the standardized initial reaction rate (Vₒ) of Dicer converting pre-miRNA into mature miRNA at various concentration of peptide 2b.

FIG. 4 is a graph representing the amount of mature miRNA produced in the presence of peptide 2b, 2c or 1e using northern blotting method.

### DETAILED DESCRIPTION OF THIS INVENTION

Features and advantages of the present invention will be more clearly understood by the following detailed description of the present preferred embodiments by reference to the accompanying drawings. It is first noted that terms or words used herein should be construed as meanings or concepts corresponding with the technical sprit of the present invention, based on the principle that the inventor can appropriately define the concepts of the terms to best describe his own invention. Also, it should be understood that detailed descriptions of well-known functions and structures related to the present invention will be omitted so as not to unnecessarily obscure the important point of the present invention.

Hereinafter, the present invention will be described in more detail.

The present invention provides an amphiphilic peptide library including one or more amphiphilic alpha helical peptide, wherein the alpha helical peptide has 4 to 12 Leu (leucine, L) at one, and of the hydrophobic amino acids, one or more Leu residues are substituted by Try (Trptophan, W).

The amphiphilic peptide library preferably contains one or more amphiphilic peptides which have the amino acid sequences arranged with the hydrophobic amino acid leucine (L) and the hydrophilic amino acid lysine (K) or glycine (G) alternately by ones or twos where two lysines (K) are substituted with two tryptophans (T), but is not limited thereto.

The amphiphilic peptide library preferably has one or more peptides having one or more of the amino acid sequences represented by SEQ ID NO: 2-SEQ ID NO: 9, or SEQ ID NO: 12-SEQ ID NO: 21, more preferably, having one or more of the amino acid sequences represented by SEQ ID NO: 12-SEQ ID NO: 21, most preferably amino acid sequences represented by SEQ ID NO 13, 16 and 18, but not always limited thereto.

The amphiphilic peptide library preferably binds specifically to the hairpin-shaped pre-miRNA and induces the production of mature miRNA (microRNA) by activating the pre-miRNA processing by Dicer enzyme, but is no limited thereto.

The miRNA is one selected from the group consisting of let-7a-1, miR16-1 and miR24-1, but is not limited thereto.

In order to prepare a peptide that specifically binds with hairpin-shaped target pre-miRNA, from the alpha helical amphiphilic peptide composed of lysine (K), leucine (L) and glycine (G), the two hydrophobic amino acids leucines (L) were substituted with tryptophan (W) having an indole group which can interact with RNA bases.

A scanning library was constructed (see Table 1) to identify the tryptophan site which is important for promoting strong and specific binding. The binding affinity of the first generation peptide, constructed by replacing one leucine to tryptophan against pre-let7a-1 and pre-miR16-1 (see Table 2) was determined by fluorescent anisotropy method.

As a result, there was a significant increase in binding affinity for pre-miRNA when leucines at #1 or #14 (1a and 1h) of amphiphilic peptide (SEQ ID NO 1) were substituted with tryptophan. Amphiphilic peptide with each ends substituted with tryptophan showed an increase in binding affinity for pre-miRNA. However, there was no effect on increasing the binding specificity against target pre-miRNA (see Table 3).

Therefore, amphiphilic peptide having substation of one tryptophan showed increase in binding affinity but not the selectivity remained unchanged.

Based on the first generation peptide, the tryptophan scan library, the present inventors constructed the second generation peptide library, having 2 tryptophan substitutions. In order to have binding affinity and specificity to the amphiphilic peptide, one of the leucine at positions 1 or 14 was substituted to tryptophan and one of the six leucines in the center region was substituted to tryptophan (Table. 4). When the binding affinity of second generation peptide against pre-let7a-1 and pre-miR16-1 using fluorescence anisotropy was measured, peptide 2b showed strongest binding with pre-let7a-1. Peptide 2b and 2c showed the first and second strongest binding with pre-miR16-1 (Table 5). When the discrimination ratio was calculated from dissociation constant, peptide 2b showed strongest binding affinity with pre-let7a-1. However, peptide 2b showed relatively weaker binding with other hairpin shape pre-miRNA, showing a high discrimination ratio of 11. Peptide 2e showed strong and specific binding with pre-miR16-1 with the discrimination ratio of 2.3. The peptide which showed strong and specific binding to pre-miR124-1 were peptide 2g and the discrimination ratio was 2.3 indicating specific recognition. Two leucine residues were replaced with tryptophan, and this peptide containing two tryptophan interacted strong and specifically with target pre-miRNA.

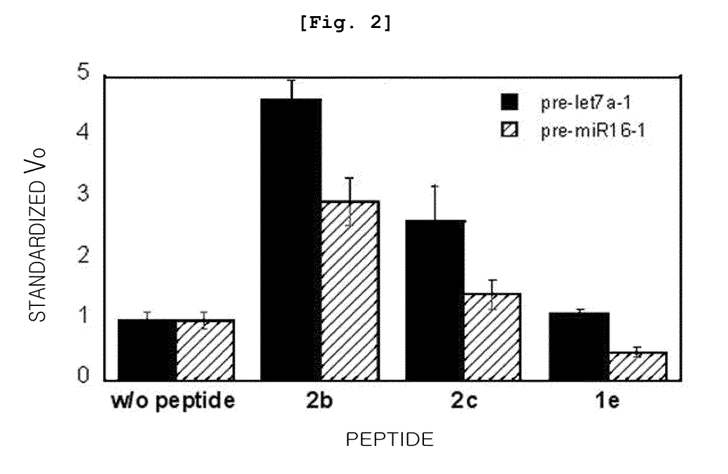

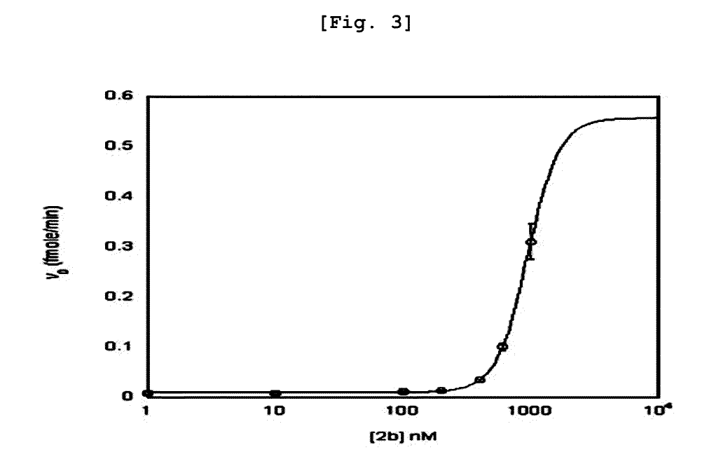

To investigate the effect of peptide 2b and 2c on Dicer enzyme activity, the present inventors measured the initial reaction velocity from the amount of reaction product by treating with isotope labeled pre-miRNAs, pre-let7a-1 or pre-miR16-1 in the presence of peptide 2b or 2c. As a result, when compared to the peptide non treated group, the processing of pre-miRNA into mature miRNA by Dicer was accelerated when treated with peptide 2b or 2c. Especially, there was a profound increase of mature miRNA production when pre-let7a-1 and 2b interacted (see FIG. 2), suggesting a strong and specific binding between the peptide and pre-miRNA. In addition, when we investigated the production of mature miRNA relative to the concentration of peptide 2b, there was a 2b concentration dependent change in initial reaction response and a rapid increase of reaction at concentrations higher than 100 nM (see FIG. 3).

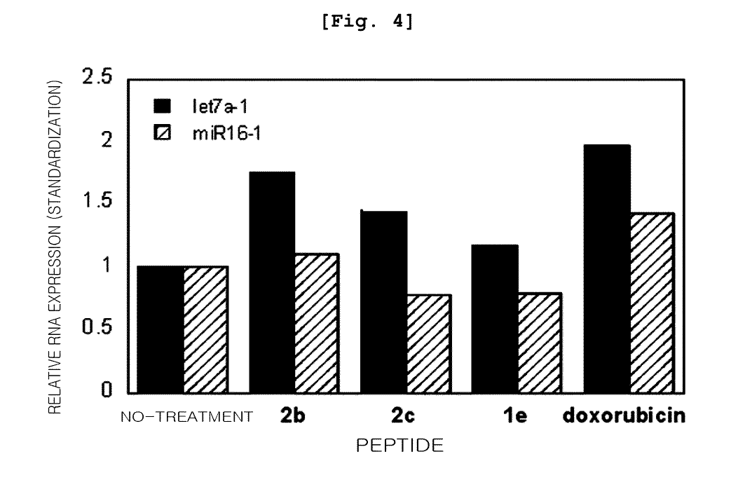

In order to study the effect of the peptide on increasing the production of mature miRNA by activating the Dicer enzyme activity on cellular level, the present inventors treated the colon cancer cell lines with peptide 2b or 2c and quantified the amount of mature miRNA production using northern blotting. The result showed an increase of target miRNA production when cells were treated with peptide. There was a significant increase of mature let7a-1 when treated with 2b, which was proved to have a specific binding affinity with pre-let7a-1 (see FIG. 4). This findings show that peptide that specifically bind to pre-miRNA can specifically induce the production of mature target miRNA.

Therefore, the amphiphilic peptide library containing two tryptophan residues can be effectively used for promoting the production of target miRNA.

The present invention provides a method for detecting an amphiphilic peptide that binds specifically to a hairpin-shaped target pre-miRNA, including:

1) preparing the amphiphilic peptide library;
2) synthesizing a hairpin-shaped target pre-miRNA and one or more hairpin-shaped non target pre-miRNAs as a control;
3) measuring a binding affinity of the amphiphilic peptide for the pre-miRNA after combining the amphiphilic peptide, the hairpin-shaped target or non target pre-miRNA, and a probe molecule; and
4) selecting an amphiphilic peptide having a stronger binding affinity for target pre-miRNA than the non target pre-miRNA.

According to the above method, the pre-miRNA in step 2) is one selected from the group consisting of pre-let-7a-1, pre-miR16-1 and pre-miR24-1, but is not limited thereto.

According to the above method, the probe molecule in step 3) is a compound having fluorescence and labeled with a tag which competes with amphiphilic peptide against binding with target hairpin-shaped pre-miRNA, but is not limited thereto.

According to the method, the binding affinity in step 3) is measured by fluorescence anisotropy using competitive binding method, but is not limited thereto.

To acquire strong and selective binding against hairpin-shaped target pre-miRNA, the amphiphilic peptide of the present invention possessed two tryptophan residues having indole moieties to increase the selection from the groove forming the double helical groove of the hairpin pre-miRNA. The constructed amphiphilic peptide showed strong and specific binding to the target pre-miRNA. The specific binding affinity improved Dicer enzyme activity and specifically increased the production of mature target miRNA. Therefore, changing the amphiphilic character of the amphiphilic peptide could be a useful method for detecting peptides that binds strongly and selectively to target pre-miRNAs.

The present invention also provides a method for detecting a pre-miRNA which specifically binds to an amphiphilic peptide, including:

1) preparing the amphiphilic peptide library;
2) synthesizing a hairpin-shaped target pre-miRNA to be detected and one or more hairpin-shaped non target pre-miRNAs as a control to screen the amphiphilic library;
3) measuring a binding affinity of pre-miRNA for amphiphilic peptide after combining the amphiphilic peptide, the hairpin-shaped target or non target pre-miRNA, and a probe molecule; and
4) selecting the target pre-miRNA having stronger binding affinity to the amphiphilic peptide than the non target pre-miRNA.

According to the above method, the probe molecule in step 3) is a compound having fluorescence and labeled with a tag which competes with amphiphilic peptide against binding with target hairpin-shaped pre-miRNA, but is not limited thereto.

According to the above method, the binding affinity in step 3) is measured by fluorescence anisotropy using competitive binding method, but is not limited thereto.

To acquire strong and selective binding against hairpin-shaped target pre-miRNA, the amphiphilic peptide of the present invention possessed two Trp residues having indol moieties to increase the selection from the groove forming the double helix groove of the hairpin pre-miRNA. The constructed amphiphilic peptide showed strong and specific binding to the target pre-miRNA. The specific binding affinity improved Dicer enzyme activity and specifically increased the production of mature target miRNA, therefore, could be a used for detecting the miRNA, which is a target for amphiphilic peptide.

The present invention also provides a method for detecting an amphiphilic peptide which increases a production of a target miRNA, including:

1) preparing the amphiphilic peptide library;
2) culturing a cell line after being treated with the amphiphilic peptide;
3) measuring the expression level of a target miRNA from the cell line; and
4) selecting an amphiphilic peptide which increases an expression level of the target miRNA compared to the non-treated control group.

It is preferable that the amphiphilic peptide binds specifically to the hairpin-shaped pre-microRNA (pre-miRNA), therefore promoting the production of mature microRNA (miRNA) by activating the Dicer enzyme, but is not limited thereof.

According to the above method, the cell line in step 2) is preferably colon cancer cell but is not limited thereto.

According to the above method, the miRNA in step 3) is one selected from the group consisting of pre-let-7a-1, pre-miR16-1 and pre-miR24-1, but is not limited thereto.

According to the above method, the expression level of miRNA in step 3) is one selected from the group consisting of northern blotting, RT-PCR and microarray, but is not limited thereto.

To acquire strong and selective binding against hairpin-shaped target pre-miRNA, the amphiphilic peptide of the present invention with two tryptophan residues of indole groups to increase the selection from the groove forming the double helix groove of the hairpin pre-miRNA. The constructed amphiphilic peptide showed strong and specific binding to the target pre-miRNA. The specific binding affinity improved Dicer enzyme activity and specifically increased the production of mature target miRNA. Therefore, changing the amphiphilic character of the amphiphilic peptide could be a useful method for detecting peptides that binds strongly and selectively to target pre-miRNAs.

In addition, the present invention provides a composition promoting the production of target miRNA specific amphiphilic peptide, which includes the amphiphilic peptide as an active compound.

The present invention also provides an application of the amphiphilic peptide for preparing a composition for promoting the production of amphiphilic peptide specific target miRNA.

The miRNA is one selected from the group consisting of let-7a-1, miR16-1 and miR24-1, but is not limited thereto.

The amphiphilic peptide of the present invention was substituted by two tryptophan residues having indole groups to increase the selectivity against the double helical groove of the hairpin pre-miRNA. The constructed amphiphilic peptide shows strong and specific binding to the target pre-miRNA, which leads to an improved Dicer enzyme activity, thus could be effectively used in increasing the production of mature target miRNA.

The present invention also provides a method for increasing the production of amphiphilic peptide specific target miRNA, including administrating the amphiphilic peptide to a subject.

The miRNA is one selected from the group consisting of let-7a-1, miR16-1 and miR24-1, but is not limited thereto.

The subject is preferably mammals, more preferably experimental animals include mice, rabbits, guinea pigs, hamsters, dogs and cats, most preferably primates, such as chimpanzees and gorillas.

The amphiphilic peptide of the present invention was substituted by two tryptophan residues having indole groups to increase the selectivity against the double helical groove of the hairpin pre-miRNA. The constructed amphiphilic peptide shows strong and specific binding to the target pre-miRNA, which leads to an improved Dicer enzyme activity, thus could be effectively used in increasing the production of mature target miRNA.

The present invention also provides a therapeutic drug or diagnostic reagent for disease caused by the inhibition of amphiphilic peptide specific target miRNA, which includes the amphiphilic peptide as an active compound.

The present invention also provides a treatment method for disease caused by the inhibition of amphiphilic peptide specific target miRNA, including administrating the amphiphilic peptide to a subject.

The present invention also provides an application of the amphiphilic peptide for producing therapeutic drug or diagnostic reagent for disease caused by the inhibition of amphiphilic peptide specific target miRNA.

The miRNA is one selected from the group consisting of let-7a-1, miR16-1 and miR24-1, but is not limited thereto.

The disease is one selected from the group consisting of colon cancer, prostate cancer, testicular cancer, small intestine cancer, colorectal cancer, anal cancer, esophageal cancer, pancreatic cancer, gastric cancer, renal cancer, cervical cancer, breast cancer, lung cancer, ovarian cancer and leukemia, but is not limited thereto.

The subject is preferably mammals, more preferably experimental animals include mice, rabbits, guinea pigs, hamsters, dogs and cats, most preferably primates, such as chimpanzees and gorillas.

The amphiphilic peptide of the present invention was substituted by two tryptophan residues having indole groups to increase the selectivity against the double helical groove of the hairpin pre-miRNA. The constructed amphiphilic peptide shows strong and specific binding to the target pre-miRNA, which leads to an improved Dicer enzyme activity, thus could be effectively used for producing therapeutic drug or diagnostic reagent and for the treatment of disease caused by the inhibition of amphiphilic peptide specific target miRNA.

The therapeutic drug may contain one or more active compounds with same or similar function, in addition to the amphiphilic peptide.

The therapeutic drug may include pharmaceutically acceptable additives, for example, starch, gelatin, micocrystalline cellulose, taffy, lactose, povidone, colloidal silicon dioxide, calcium phosphate, mannitol, gum acacia, pregelatinized starch, corn starch, cellulose powder, hydroxypropyl cellulose, opadry, sodium starch glycolate, carnauba, Pb, synthetic aluminum silicate, stearic acid, magnesium stearate, aluminum stearate, calcium stearate, white sugar, dextrose, sorbitol and talc. The pharmaceutically acceptable additives are contained in an amount of 0.01-90 wt %, but are not limited thereto.

The pharmaceutical composition of the present invention may be formulation for oral or parenteral administration. When converting the pharmaceutical composition into administration forms, fillers, extenders, binders, wetting agents, disintegrating agents or surfactants are used.

Formulations for oral administration include, for example, tablets, pills, granules and capsules. Solid oral formulation may contain at least one or more diluents, such as starch, calcium carbonate, sucrose, lactose or gelatin. Solid oral formulation may also contain lubricants, such as magnesium stearate and talc. The liquid oral formulation may include suspensions, emulsions, syrups and elixirs. These formulations may contain conventional diluents, for example water and liquid paraffin, or other diluents, such as adsorption agents, colorants, flavoring agents, perfumes and preservatives. Formulation for parenteral administration includes sterile aqueous solution, non-aqueous solution, suspension, emulsion, lyophilized formulations and suppositories. As a solvent for non-aqueous solution and suspension, propylene glycol, polyethylene glycol, vegetable oil such as olive oil, or injectable ester such as ethyolate may be used. As a base for suppositories, Witepsol, Macrogol, Tween 61, cacao fat, laurin fat, glycerogelatin, etc. may be used.

The pharmaceutical composition according to the present invention may be administered to a subject in an effective dose of 0.0001˜100 mg/kg, preferably 0.001˜10 mg/kg, once to several times per day. The dosage can be varied considering various factors including age, weight, health condition, gender and dietary habit, administration frequency and pathway, excretion and severity of the disease.

The administration pathway of the therapeutic drug may be by oral administration or parenteral administration. The drug may be administrated in the form of conventional pharmaceutical composition parenterally, in particular by intraperitoneal injection, intrarectal injection, subcutaneous injection, intraveouns injection, intramuscular injection, intrammamry injection, cerebral vascular injection or intrathoracic injection.

The therapeutic drug may be use alone, or combined with other treatment methods such as, surgery, radiotherapy, hormone therapy, chemotherapy and biological response modifiers.

Additionally, the present invention provides a method for promoting in vitro target pre-miRNA processing, including:

1) treating an amphiphilic peptide selected by the above method with pre-miRNA present in in vitr; and

2) binding the amphiphilic peptide and the target pre-miRNA.

According to the above method, the pre-miRNA in step 1) is one selected from the group consisting of pre-let-7a-1, pre-miR16-1 and pre-miR24-1, but is not limited thereto.

The amphiphilic peptide of the present invention was substituted by two trptophan residues having indole groups to increase the selectivity against the double helical groove of the hairpin pre-miRNA. The constructed amphiphilic peptide shows strong and specific binding to the target pre-miRNA, which leads to an improved Dicer enzyme activity, thus could be effectively used in increasing the processing of mature target miRNA in vitro.

### EXAMPLES

Hereinafter, the present invention will be described in more detail with reference to the following examples and experimental examples. However, the following examples and experimental examples are provided for illustrative purposes only, and the scope of the present invention should not be limited thereto in any manner.

### Example 1

### Synthesis of First Generation Peptide

Peptide was synthesized using the general solid phase synthesis method using link amide MBHA resin (0.4˜0.6 mmol/g) at a 25 μmol scale. The amino acid monomer used for synthesis was purchased from NovaBiochem. The N-terminus of the peptides were acetylated, the peptides were detected by Auto Flex MALDI-TOF/TOF mass spectrometer (Bruker Daltonics, Germany) equipped with 337 mM of nitrogen laser and 1.2 m flight tube. Also, the peptide was isolated and its purity was confirmed by Agilent 1100 HPLC (high performance liquid chromatography.

In detail, peptide 1 shown in Table 1 was synthesized with 50 mg (32 μmol) of link amide MBHA resin (0.64 mmol/g). The resin underwent swelling with dichloromethane (1 ml, 5 min) and dimethylformamide (DMF, 1 ml, 5 min) and then stirred twice for 5 min with 1.5 ml of DMF containing 20% of piperidine to remove the Fmoc protective groups. Piperidine solution was completely removed by stirred with dichloromethane (1 ml, 5 times), DMF (1 ml, 2 times) and followed by filtration. In peptide 1, the first amino acid Glycine was attached by dissolving the Fmoc protected monomer, FmocGly-OH (48 mg, 5 eq)(benzotriazol-1-yl-oxy-tris-pyrridino-phosphonium hexafluoro-phosphate, PyBOP, 84 mg, 5 eq) in 1 ml of DMF, and then N,N-diisopropylethylamine (DIPEA, 56 μl, 12 eq) was added. This solution was added to the resin where Fmoc protective resin was removed and reacted at room temperature for 1 hr using rotating stirrer. The termination of the reaction was confirmed by 2,4,6-trnitrobenzenesulfonic acid (TNBS) test. Following the reaction termination, the reaction solution was removed by stirring with dichloromethane (1 ml, 5 times), DMF (1 ml, 2 times) and followed by filtration. As described above, removal of Fmoc and binding of amino acid was repeated until the last amino acid was attached. Acetylation of N-terminus was removed and N-Hydroxybenzotriazole (HOBt, 41.2 mg, 10 eq), anhydrous acetic acid (29 μl) were dissolved in 900 μl DMF and 100 μl dichloromethane. This solution was added to the resin and stirred for 1 hr at room temperature. To separate the synthesized peptide from the solid phase, 1.5 ml of cleave cocktail {trifluoroacetic acid, (TFA)/triisopropyl silane (TIS)/water=95:2.5:2.5} was added and stirred for 2 hrs. After stirring, the resin was removed by filtration, washed with trifluoroacetic acid (TFA) (0.5 ml, 2 times) and the remaining peptide was collected. The solution yielded was concentrated under nitrogen, then the peptide was precipitated with cold diethyl ether and n-hexane (v/v=50/50). The precipitates were centrifuged at 13,000 rpm for 5 min to collect as a pellet, and the supernatant was carefully removed by decantation. This pellet was centrifuged two times with 10 ml of cold diethyl ether and n-hexane (v/v=50/50) and washed by decantation. The pellet obtained was air dried and dissolved with 0.2 ml of dimethyl sulfoxide (DMSO) and HPLC purification was performed by passing through a 0.45 μm syringe filter. Waters 600 HPLC was used and for the stationary phase, XBridge™ Prep C18 5 μm OBD™ (19 mm×150 mm, Waters) column was used. For the mobile phase, gradient between Buffer A, water containing 0.1% TFA, and buffer B, CH₃CN containing 0.1% TFA was used for isolation. Peptide 1 was isolated from 30˜35% of gradient B condition, the peptide was lyophilized and yielded as white powder (4.8 mg, yield 8%). All other peptides were acquired according to the method described above.

The result of calculated mass and the actual mass analyzed by MALDI-TOF mass spectrometry on amino acid sequence of peptide 1 and peptide (1a-1h) which were tryptophan scanned instead of the leucine are shown in Table. 1.

TABLE 1

| | | calculated | Actual |
|---|---|---|---|
| Peptide | Sequence | mass^{a} | mass |
| 1 | Ac-LKKLLKLLKKLLKLAG | 1861.5 | 1861.5 |
| | (SEQ ID NO. 1) | | |
| 1a | Ac-WKKLLKLLKKLLKLAG | 1934.3 | 1934.3 |
| | (SEQ ID NO. 2) | | |
| 1b | Ac-LKKWLKLLKKLLKLAG | 1934.3 | 1934.4 |
| | (SEQ ID NO. 3) | | |
| 1c | Ac-LKKLWKLLKKLLKLAG | 1934.3 | 1934.4 |
| | (SEQ ID NO. 4) | | |
| 1d | Ac-LKKLLKWLKKLLKLAG | 1934.3 | 1934.3 |
| | (SEQ ID NO. 5) | | |
| 1e | Ac-LKKLLKLWKKLLKLAG | 1934.3 | 1934.5 |
| | (SEQ ID NO. 6) | | |
| 1f | Ac-LKKLLKLLKKWLKLAG | 1934.3 | 1934.6 |
| | (SEQ ID NO. 7) | | |
| 1g | Ac-LKKLLKLLKKLWKLAG | 1934.3 | 1934.1 |
| | (SEQ ID NO. 8) | | |
| 1h | Ac-LKKLLKLLKKLLKWAG | 1934.3 | 1934.1 |
| | (SEQ ID NO. 9) | | |

^{a}The calculated mass of each peptide is? (M + H⁺).

### Example 2

### Synthesis of Pre-miRNA

The RNA used to measure the binding affinity with the peptides synthesized in <Example 1> were synthesized in large scale by in vitro transcription using T7 polymerase. A 5′ linker (5′-GGGAGA-3′) was added for T7 polymerase activity. The base sequences are shown in Table. 2.

TABLE 2

| pre-miRNA | Base sequence(5′-3′) |
|---|---|
| pre-let7a-1 | GGGAGAUGAGGUAGUAGGUUGUAUAGUUUUAGGGUCACA |
| | CCCACCACUGGGAGAUAACUAUACAAUCUACUGUCUUUC |
| | (SEQ ID NO. 10) |
| pre-miR16-1 | GGGAGACAGCACGUAAAUAUUGGCGUUAAGAUUCUAAAA |
| | UUAUCUCCAGUAUUAACUGUGCUGCUGAA |
| | (SEQ ID NO. 11) |

### Example 3

### Determination of the Dissociation Constant Between First Generation Peptide and the Pre-miRNA

The binding affinity between the peptide synthesized in <Example 1> and pre-miRNA from <Example 2> were measured by fluorescence anisotropy using competitive binding method. A rev peptide with rhodamine attached at the N-terminal was used as a fluorescent probe and LS-55 luminescence spectrometer from Perkin-Elmer was use for analysis. A constant temperature of 20° C. was maintained by a water bath. The fluorescent intensity was measured by of 200 nM of rhodamine-rev peptide was measured with an excitation wavelength at 550 nm (band path 10 nm) and detection at 580 nm (band path 10 nm). The integration time was sec. Each experimental value is the mean value of 5 measurements. A 20 mM of HEPES{4-(2-hydroxyethyl)-1-piperazineethanesulfonic acid} buffer (pH 7.5) containing 140 mM NaCl, 5 mM KCl and 1 mM MgCl₂ was used for measuring the dissociation constant. The dissociation constant was determined according to the Mathematical Formula 1 and using Kaledia graph.

$$A = {A}_{0} + \frac{\begin{matrix}Δ A ( {[ RNA ]}_{0} + {[ Rh - rev ]}_{0} + {K}_{d} ) - \\ ( \sqrt{\begin{matrix}{( {[ RNA ]}_{0} + {[ Rh - rev ]}_{0} + {K}_{d} )}^{2} - \\ {{4 [ RNA ]}_{0} [ Rh - rev ]}_{0}\end{matrix}} )\end{matrix}}{{2 [ Rh - rev ]}_{0}} \qquad [ Mathematical Formula 1 ]$$

A and Aₒ are the fluorescence anistropy in the presence or absence of RNA, whereas ΔA is the difference between these two values. [RNA]₀ and [Rh-rev]₀ are initial concentrations of RNA and rev peptide binding with rhodamine.

When the dissociation constant between peptide synthesized in <Example 1> and one of two pre-miRNAs (pre-let7a-1, pre-miR16-1) synthesized in <Example 2> were analyzed, as shown in Table 3, substitution of one peptide into tryptophan increased the dissociation by 150 times (peptide 1h) compared to the peptide with no substitution (peptide 1). The increase in dissociation constant was due to the changes on both terminus of the peptide, indicating the importance of each terminus for tryptophan substitution (Table 3).

TABLE 3
Dissociation constant K_{d} ^{a} between first generation
peptide and pre-let7a-1 or pre-miR16-1

| | | K_{d} value with | K_{d} value with |
|---|---|---|---|
| Pep- | | pre-let7a-1 | pre-miR16-1 |
| tide | Amino acid sequence | (nM) | (nM) |
| 1 | Ac-LKKLLKLLKKLLKLAG | 17 | 12 |
| | (SEQ ID NO. 1) | | |
| 1a | Ac-WKKLLKLLKKLLKLAG | 0.17 | 0.42 |
| | (SEQ ID NO. 2) | | |
| 1b | Ac-LKKWLKLLKKLLKLAG | 0.68 | 1.2 |
| | (SEQ ID NO. 3) | | |
| 1c | Ac-LKKLWKLLKKLLKLAG | 0.41 | 0.56 |
| | (SEQ ID NO. 4) | | |
| 1d | Ac-LKKLLKWLKKLLKLAG | 0.64 | 1.8 |
| | (SEQ ID NO. 5) | | |
| 1e | Ac-LKKLLKLWKKLLKLAG | 20 | 27 |
| | (SEQ ID NO. 6) | | |
| 1f | Ac-LKKLLKLLKKWLKLAG | 0.32 | 0.48 |
| | (SEQ ID NO. 7) | | |
| 1g | Ac-LKKLLKLLKKLWKLAG | 0.25 | 0.56 |
| | (SEQ ID NO. 8) | | |
| 1h | Ac-LKKLLKLLKKLLKWAG | 0.11 | 0.4 |
| | (SEQ ID NO. 9) | | |

^{a} K_{d} value was determined by competitive fluorescence anisotropy method using fluorescence labeled rhodamine-rev peptide.

### Example 4

### Synthesis of Second Generation Peptide Having Increased Binding Specificity

In order to synthesize an improved peptide than the tryptophan scanning peptides from <Example 1>, second generation peptides were synthesized, in which one of the leucine at the terminus is substituted to tryptophan and one of the six internal leucines is substituted to tryptophan. The peptides were designed to increase the binding affinity when substituted by tryptophan at both terminus, and to increased binding specificity when substituted by tryptophan in the center region. The second generation peptide was designed and synthesized based on the result from <Example 1>, in which the peptides with tryptophan substitution increased the binding affinity (Table 4).

Table 4 below shows the amino acid sequence of the second generation peptide, calculated mass and the actual mass analyzed by MALDI-TOF mass spectrometry.

TABLE 4

| | | Calculated | Actual |
|---|---|---|---|
| Peptide | Amino acid sequence | Mass^{a} | Mass |
| 2a | Ac-WKKLWKLLKKLLKLAG | 2007.3 | 2007.8 |
| | (SEQ ID NO. 12) | | |
| 2b | Ac-WKKLLKWLKKLLKLAG | 2007.3 | 2007.8 |
| | (SEQ ID NO. 13) | | |
| 2c | Ac-WKKLLKLLKKWLKLAG | 2007.3 | 2007.7 |
| | (SEQ ID NO. 14) | | |
| 2d | Ac-WKKLLKLLKKLWKLAG | 2007.3 | 2007.6 |
| | (SEQ ID NO. 15) | | |
| 2e | Ac-WKKLLKLLKKLLKWAG | 2007.3 | 2007.8 |
| | (SEQ ID NO. 16) | | |
| 2f | Ac-LKKLWKLLKKLLKWAG | 2007.3 | 2007.6 |
| | (SEQ ID NO. 17) | | |
| 2g | Ac-LKKLLKWLKKLLKWAG | 2007.3 | 2007.7 |
| | (SEQ ID NO. 18) | | |
| 2h | Ac-LKKLLKLLKKWWKLAG | 2007.3 | 2007.9 |
| | (SEQ ID NO. 19) | | |
| 2i | Ac-LKKLLKLLKKWLKWAG | 2007.3 | 2007.8 |
| | (SEQ ID NO. 20) | | |
| 2j | Ac-LKKLLKLLKKLWKWAG | 2007.3 | 2007.6 |
| | (SEQ ID NO. 21) | | |

^{a}The calculated mass of each peptide is (M + H⁺).

### Example 5

### Determination of Dissociation Constant and Specificity of Second Generation Peptide and Pre-miRNA

According the method described in <Example 3>, the dissociation constant between second generation peptide and 2 pre-miRNAs synthesized from <Example 2> was determined using fluorescent polarization detection method and fluorescent probe molecule.

The result in Table 5 indicates that the second generation peptides show strong and specific binding with pee-miRNA with a hairpin-shaped structure. Peptide 2b showed strong binding with pre-let7a-1RNA, in particular. There was a significant increase in binding affinity in second generation peptide with 2 tryptophan substitution (260 times higher than non tryptophan substitution), which is due to better discrimination among other RNAs (discrimination ratio increased from 1 to 11).

TABLE 5
K_{d}^{a} values of second generation peptides against
pre-let7a-1 and pre-miR16-1.

| | | K_{d} value | K_{d}value |
|---|---|---|---|
| | | against | against |
| | | pre-let7a- | pre-miR16- |
| peptide | Amino acid sequence | 1 (nM) | 1 (nM) |
| 1 | Ac-LKKLLKLLKKLLKLAG | 17 | 12 |
| 2a | Ac-WKKLWKLLKKLLKLAG | 0.23 | 0.32 |
| 2b | Ac-WKKLLKWLKKLLKLAG | 0.067 | 0.22 |
| 2c | Ac-WKKLLKLLKKWLKLAG | 0.15 | 0.27 |
| 2d | Ac-WKKLLKLLKKLWKLAG | 0.25 | 0.64 |
| 2e | Ac-WKKLLKLLKKLLKWAG | 0.22 | 0.28 |
| 2f | Ac-LKKLWKLLKKLLKWAG | 0.90 | 2.1 |
| 2g | Ac-LKKLLKWLKKLLKWAG | 0.96 | 1.1 |
| 2h | Ac-LKKLLKLLKKWWKLAG | 1.5 | 2.6 |
| 2i | Ac-LKKLLKLLKKWLKWAG | 0.31 | 0.47 |
| 2j | Ac-LKKLLKLLKKLWKWAG | 0.37 | 0.44 |

^{a}K_{d} value was determined by competitive fluorescence anisotropy method using fluorescence labeled rhodamine-rev peptide.

As shown in Table 6, the discrimination ratio (mean value of K_{d} against all hairpin pre-miRNA/K_{d} value against target pre-miRNA) of petide2b/pre-let7a-1, which compares the binding affinity (K_{d} value) of one of the second generation peptide, peptide 2b against target hairpin RNA, pre-let7a-1 was 11. Higher discrimination ratio means a peptide-RNA binding having specific recognition, suggesting that the binding between peptide 2b/hairpin RNA pre-let7a-1 is very specific. Peptide 2b also forms the strongest binding with peptide 2b is as strong as 250 pM and the discrimination ratio of peptide 2e and pre-miR16-1 against other RNAs were 2.3, suggesting a strong and specific interaction between them. In case of pre-miR24-1, the discrimination ratio of 2 g/pre-miR24-1 was 2.3, suggesting a specific recognition.

TABLE 6
The comparison of K_{d}^{a} values of second generation peptides against
pre-s miRNA or other hairpin RNAs.

| | K_{d} (nM)^{b} | | | | |
|---|---|---|---|---|---|
| Peptide | pre-let7a-1 | pre-miR16-1 | pre-miR24-1 | HBV | IRES |
| 2a | 0.23 (2.8) | 0.32 (2.6) | 0.82 (0.66) | 0.77 | 0.90 |
| 2b | 0.067 (11) | 0.22 (1.1) | 0.43 (1.5) | 0.90 | 1.0 |
| 2c | 0.15 (2.8) | 0.27 (1.4) | 0.88 (0.41) | 0.46 | 0.46 |
| 2d | 0.22 (1.2) | 0.64 (1.2) | 0.67 (1.0) | 0.79 | 0.77 |
| 2e | 0.90 (1.7) | 0.28 (2.3) | 0.84 (0.50) | 0.34 | 0.76 |
| 2f | 0.96 (9.3) | 2.1 (1.2) | 2.2 (0.66) | 0.38 | 3.8 |
| 2g | 0.96 (0.85) | 1.1 (0.99) | 0.47 (2.3) | 1.0 | 1.3 |
| 2h | 1.5 (1.8) | 2.6 (1.0) | 1.5 (1.9) | 5.7 | 1.7 |
| 2i | 0.31 (4.4) | 0.47 (1.6) | 0.53 (1.2) | 0.82 | 1.2 |
| 2j | 0.37 (1.0) | 0.44 (1.4) | 0.27 (1.9) | 0.35 | 0.85 |

^{a}K_{d} value was determined by competitive fluorescence anisotropy method using fluorescence labeled rhodamine-rev peptide.
^{b}Discrimination ratio against each RNA is indicated in parenthesis. Discrimination ratio is defined as K_{d} value against other RNA (HBV, IRES, other miRNAs)/K_{d} value against target miRNA.

### Example 6

### Dicer Activity Assay in the Presence of Selected Amphiphilic Peptide

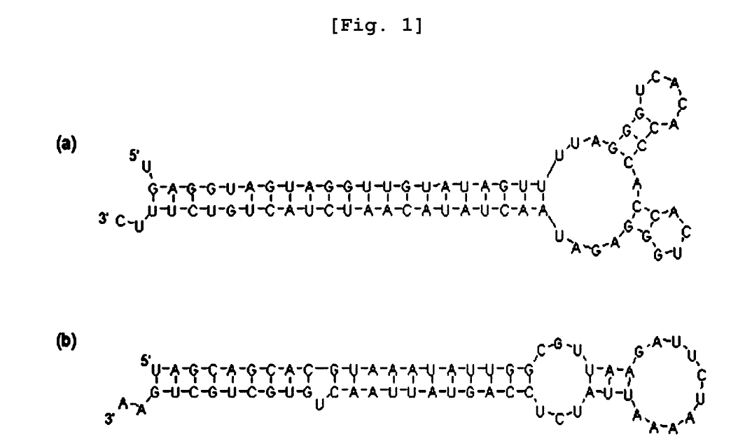

Two types of pre-miRNA (pre-let7a-1, pre-miR16-1) used as the substrate for the Dicer enzyme activity assay was ordered and manufactured by ST Pharm Co., Ltd (FIG. 1). For Dicer activity assay, the 5′ end of the synthesized pre-miRNA was labeled with ³²p isotope. Two pmole of each pre-miRNA was isotope labeled with 20 mCi of [g-³²P]ATP (New England Biolabs) using 10 units of T4 polynucleotide kinase (New England Biolabs). Total volume of 10 μl was isotope labeled for 1 hr at 37° C. The isotope labeled RNA obtained after the reaction was purified using G-25 Sephadex column (Sigma). The purified substrate was used for measuring Dicer enzyme activity. Total reaction mixture of 30 μl containing 1 nM of isotope labeled pre-miRNA and 0.001 u/μl of recombinant human Dicer (Genlantis, CA, USA) were incubated at 37° C. in 24 mM 4-(2-hydroxyethyl)-1-piperazineethanesulfonic acid, HEPES) buffer system containing 200 mM NaCl, 0.04 mM EDTA, 1 mM ATP and 2.5 mM MgCl₂(pH 8.0). After reacting for 0, 10, 30, 60 or 120 min in the presence of 400 nM concentration of 2b or 2c peptide, 3 μl of each reaction mixture was mixed with 5 μl of RNA gel loading buffer {95% formamide, 10 mM ethylenediaminetetraacetic acid (EDTA), 0.05% FF (xylene cyanol FF)} and incubated for 5 min at 65° C. The amount of the reaction product was determined by gel running in 15% polyacrylamide gel containing 7M urea, and then the initial reaction velocity (V₀) from each reaction was compared. The initial reaction velocity (V₀) of the enzyme is the reaction velocity of the initial linear time (until 30 min). Each initial velocity was normalized with the initial velocity from each substrate condition without peptide treatment. The initial reaction velocity of Dicer for pre-let7a-1 and pre-miR16-1 without peptide treatment were 0.0071 fmole/ml and 0.018 fmole/min, respectively. Each of the initial reaction velocity is a mean data of more than 3 measurements, and the standard deviation is shown as error bars.

When the velocity of pre-miRNA converted into mature miRNA in the presence of 400 nM of each peptide, then standardized and shown as bar graphs as shown in FIG. 2, there was a highest acceleration of initial reaction rate of Dicer enzyme between 2b peptide and pre-let7a-1. This is related to the affinity between peptide 2b and pre-let7a-1, where as the strong the affinity, the faster processing reaction velocity of the Dicer. The processing velocity of pre-miR16-1 was significantly increased by peptide 2b, indicating that peptide 2b binds strongly to pre-miR16-1 as well. In the case of peptide 2c, there was an increase in initial reaction velocity involving affinity to pre-let7a-1 and pre-miR16-1. In contrast, peptide 1e which was used as a negative control did not increase the initial velocity of the Dicer processing reaction.

In addition, as shown in FIG. 3, the initial velocity of Dicer in processing pre-miRNA was dependent to the concentration of peptide 2b. According to the Mathematical Formula 2 below, The EC₅₀ is 940 nM and the hill slope is 3.5. There was a significant increase in initial reaction velocity when peptide 2b concentration was higher than 100 nM. There was a decrease of the initial reaction velocity when the concentration of the peptide 2b was higher than 1,000 nM.

$$Y = b + \frac{( t - b )}{1 + {10}^{( {logEC}_{50} - logX ) HillSlope}} \\ ( b ; bottom , t ; top ) \qquad [ Mathematical Formula 2 ]$$

### Example 7

### Analysis of the Production of Target miRNA in the Presence of Selected Amphiphilic Peptide

<7-1> HCT116 Cell Culture and Extraction of Total RNA

The HCT116 cell was purchased from American Type Culture Collection (ATCC) and was cultured in RPMI1640 medium at 37° C., under 5% CO₂. Total RNA was extracted using Trizol reagent (Invitrogen) according to the manufactures recommended protocol.

The change in RNA amount was analyzed by culturing the cells with 2 μM concentration of peptide 2b or peptide 2c and with lipofectamine.

<7-2> Northern Blotting

HCT116 cells were treated with 2 μM of peptide 2b, 2c, 1e or 0.2 μg/ml of doxorubicin for 3 hrs before analyzing the production of mature miRNA in total RNA using northern blotting. The 18S rRNA band intensity was used as the loading control. P53 inducer doxorubicin was shown to increase miRNA through Drosha enzyme and was used as a positive control. Peptide 1e was used as a negative control. Decade marker (Ambion) and 10˜20 μg of total RNA were separated by using 12.5% polyacrylamide gel containing 7M urea in 20 mM of 3-(N-morpholino)propanesulfonic acid (MOPS)-NaOH(pH 7.0) buffer at 350V for 2 hrs. RNA blotting was performed by chemical cross-linking method. Each of the separated RNA was transferred with the neutral nylon membrane (Hybond NX, Amersham/Pharmacia) and crosslinked to the membrane using 1-ethyl-3-(3-dimethylaminopropyl)carbodiimide (EDC). The membrane was prehybridized with the hybridization buffer (Clontech, CA, USA) containing 50 μl of 10 mg/ml of salmon sperm DNA. Next, the isotope labeled probe DNA which is complementary to the miRNA sequence was hybridized for 2 hrs in the same buffer condition.

The probe was isotope labeled with 25 μCi of [γ-³²P] ATP (New England Biolabs) and 5 units of polynucleotide kinase (New England Biolabs). The probe was ethanol precipitated and dissolved in 50 μl of DEPC treated water for use. The membrane was washed twice for 30 min with 200 ml of wash buffer containing 0.3 M NaCl, 0.03 M sodium citrate and 0.05% sodium dodecyl sulfate (SDS)(pH 7.0), then washed twice for 15 min with 200 ml of wash buffer containing 0.015 M NaCl, 0.0015 M sodium citrate and 0.1% SDS(pH 7.0). The membrane was exposed on the phosphorimager screen and each of the band intensity was detected by FLA-3000 and analyzed by using MultiGauge Ver. 3.0 software (Fuji Photo).

The result, as shown in FIG. 4 indicated a specific change in miRNA production regarding to the peptide type. There was a significant increase in let7a-1 level with peptide 2 treatment. The level of miR16-1 increased with peptide 2b treatment and the level of let7a-1 increased with peptide 2c treatment. These trends are similar to the trend of the initial velocity of the Dicer process in the presence of peptide.

An artificial peptide having specificity and strong binding strength to hairpin-shaped target pre-miRNA may be prepared by the present invention when compared to peptides in nature. This strong and specific binding induces the Dicer enzyme activity and selectively increases mature target miRNA production. The peptide selected by the above method of the present invention can be effectively used in studying the function of miRNA and developing novel therapeutic drug for treating target miRNA related diseases.

### INDUSTRIAL APPLICABILITY

As described above, the amphiphilic peptide selected from the present invention selectively induces mature miRNA production by tight and specific dissociation constant to the hairpin-shaped structure of the target miRNA. Therefore, the amphiphilic peptide could be a effectively used for developing drugs for treating target miRNA related disease and for manufacturing diagnostic reagents.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1-18. (canceled)

19. A method for detecting an amphiphilic peptide that binds specifically to a hairpin-shaped target pre-miRNA, the method comprising:
1) preparing the amphiphilic peptide library comprising one or more amphiphilic alpha helical peptide, wherein the alpha helical peptide has 4 to 12 Leu (leucine, L) at one, and of the hydrophobic amino acids, one or more Leu residues are substituted by Try (Trptophan, W);
2) synthesizing a hairpin-shaped target pre-miRNA and one or more hairpin-shaped non target pre-miRNAs as a control;
3) measuring a binding affinity of the amphiphilic peptide for the pre-miRNA after combining the amphiphilic peptide, the hairpin-shaped target or non target pre-miRNA, and a probe molecule; and
4) selecting an amphiphilic peptide having a stronger binding affinity for target pre-miRNA than the hairpin-shaped non target pre-miRNA.

20. The method according to claim 19, wherein the amphiphilic peptide library comprises one or more amphiphilic alpha helical peptide which has an amino acid sequence arranged with hydrophobic leucine (L) amino acid and hydrophilic lysine (K) or glycine (G) amino acids alternately by ones or twos where two lysines (K) are substituted with two tryptophans (A).

21. The method according to claim 19, wherein the amphiphilic peptide library comprises one or more amphiphilic alpha helical peptides having the amino acid sequences represented by SEQ ID NO: 2-SEQ ID NO: 9, or SEQ ID NO: 12-SEQ ID NO: 21.

22. The method according to claim 19, wherein the pre-miRNA in step 2) is one selected from the group consisting of pre-let-7a-1, pre-miR16-1 and pre-miR24-1.

23. The method according to claim 19, wherein the probe in step 3) is a compound with a tag capable of competing with the amphiphilic peptide for binding to a target hairpin-shaped pre-miRNA.

24. A method for detecting a pre-miRNA which specifically binds to an amphiphilic peptide, the method comprising:
1) preparing the amphiphilic peptide library comprising one or more amphiphilic alpha helical peptide, wherein the alpha helical peptide has 4 to 12 Leu (leucine, L) at one, and of the hydrophobic amino acids, one or more Leu residues are substituted by Try (Trptophan, W);
2) synthesizing a hairpin-shaped target pre-miRNA to be detected and one or more hairpin-shaped non target pre-miRNAs as a control to screen the amphiphilic library;
3) measuring a binding affinity of pre-miRNA for amphiphilic peptide after combining the amphiphilic peptide, the hairpin-shaped target or non target pre-miRNA, and a probe molecule; and
4) selecting the target pre-miRNA having stronger binding affinity to the amphiphilic peptide than the non target pre-miRNA.

25. The method according to claim 24, wherein the amphiphilic peptide library comprises one or more amphiphilic alpha helical peptide which has an amino acid sequence arranged with hydrophobic leucine (L) amino acid and hydrophilic lysine (K) or glycine (G) amino acids alternately by ones or twos where two lysines (K) are substituted with two tryptophans (A).

26. The method according to claim 24, wherein the amphiphilic peptide library comprises one or more amphiphilic alpha helical peptides having the amino acid sequences represented by SEQ ID NO: 2-SEQ ID NO: 9, or SEQ ID NO: 12-SEQ ID NO: 21.

27. The method according to claim 24, wherein the pre-miRNA in step 2) is one selected from the group consisting of pre-let-7a-1, pre-miR16-1 and pre-miR24-1.

28. The method according to claim 24, wherein the probe in step 3) is a compound with a tag capable of competing with the amphiphilic peptide for binding to a target hairpin-shaped pre-miRNA.

29. A method for detecting an amphiphilic peptide which increases a production of a target miRNA, the method comprising:
1) preparing the amphiphilic peptide library comprising one or more amphiphilic alpha helical peptide, wherein the alpha helical peptide has 4 to 12 Leu (leucine, L) at one, and of the hydrophobic amino acids, one or more Leu residues are substituted by Try (Trptophan, W);
2) culturing a cell line after being treated with the amphiphilic peptide;
3) measuring the expression level of a target miRNA from the cell line; and
4) selecting an amphiphilic peptide which increases an expression level of the target miRNA compared to the non-treated control group.

30. The method according to claim 29, wherein the amphiphilic peptide library comprises one or more amphiphilic alpha helical peptide which has an amino acid sequence arranged with hydrophobic leucine (L) amino acid and hydrophilic lysine (K) or glycine (G) amino acids alternately by ones or twos where two lysines (K) are substituted with two tryptophans (A).

31. The method according to claim 29, wherein the amphiphilic peptide library comprises one or more amphiphilic alpha helical peptides having the amino acid sequences represented by SEQ ID NO: 2-SEQ ID NO: 9, or SEQ ID NO: 12-SEQ ID NO: 21.

32. The method according to claim 29, wherein the cell line in step 2) is a colon cancer cell line.

33. The method according to claim 29, wherein the miRNA in step 3) is one selected from the group consisting of let-7a-1, miR16-1 and miR24-1.

34. The method according to claim 29, wherein the expression level of step 3) is one selected from the group consisting of northern blotting, RT-PCR and microarray.

35. A composition for promoting a production of amphiphilic peptide specific target miRNA, which comprises the amphiphilic peptide selected by the method of claim 19 as an active component.

36. A composition for promoting a production of amphiphilic peptide specific target miRNA, which comprises the amphiphilic peptide selected by the method of claim 24 as an active component.

37. A composition for promoting a production of amphiphilic peptide specific target miRNA, which comprises the amphiphilic peptide selected by the method of claim 29 an active component.


SEQUENCE LISTING (21 sequences)

SEQ ID NO: 1 - protein, 16 aa, Artificial Sequence
  peptide 1
LKKLLKLLKKLLKLAG

SEQ ID NO: 2 - protein, 16 aa, Artificial Sequence
  peptide 1a
WKKLLKLLKKLLKLAG

SEQ ID NO: 3 - protein, 16 aa, Artificial Sequence
  peptide 1b
LKKWLKLLKKLLKLAG

SEQ ID NO: 4 - protein, 16 aa, Artificial Sequence
  peptide 1c
LKKLWKLLKKLLKLAG

SEQ ID NO: 5 - protein, 16 aa, Artificial Sequence
  peptide 1d
LKKLLKWLKKLLKLAG

SEQ ID NO: 6 - protein, 16 aa, Artificial Sequence
  peptide 1e
LKKLLKLWKKLLKLAG

SEQ ID NO: 7 - protein, 16 aa, Artificial Sequence
  peptide 1f
LKKLLKLLKKWLKLAG

SEQ ID NO: 8 - protein, 16 aa, Artificial Sequence
  peptide 1g
LKKLLKLLKKLWKLAG

SEQ ID NO: 9 - protein, 16 aa, Artificial Sequence
  peptide 1h
LKKLLKLLKKLLKWAG

SEQ ID NO: 10 - RNA, 78 nt, Artificial Sequence
  pre-let7a-1
gggagaugagguaguagguuguauaguuuuagggucacacccaccacugggagauaacuauacaaucuacugucuuuc

SEQ ID NO: 11 - RNA, 68 nt, Artificial Sequence
  pre-miR16-1
gggagacagcacguaaauauuggcguuaagauucuaaaauuaucuccaguauuaacugugcugcugaa

SEQ ID NO: 12 - protein, 16 aa, Artificial Sequence
  peptide 2a
WKKLWKLLKKLLKLAG

SEQ ID NO: 13 - protein, 16 aa, Artificial Sequence
  peptide 2b
WKKLLKWLKKLLKLAG

SEQ ID NO: 14 - protein, 16 aa, Artificial Sequence
  peptide 2c
WKKLLKLLKKWLKLAG

SEQ ID NO: 15 - protein, 16 aa, Artificial Sequence
  peptide 2d
WKKLLKLLKKLWKLAG

SEQ ID NO: 16 - protein, 16 aa, Artificial Sequence
  peptide 2e
WKKLLKLLKKLLKWAG

SEQ ID NO: 17 - protein, 16 aa, Artificial Sequence
  peptide 2f
LKKLWKLLKKLLKWAG

SEQ ID NO: 18 - protein, 16 aa, Artificial Sequence
  peptide 2g
LKKLLKWLKKLLKWAG

SEQ ID NO: 19 - protein, 16 aa, Artificial Sequence
  peptide 2h
LKKLLKLLKKWWKLAG

SEQ ID NO: 20 - protein, 16 aa, Artificial Sequence
  peptide 2i
LKKLLKLLKKWLKWAG

SEQ ID NO: 21 - protein, 16 aa, Artificial Sequence
  peptide 2j
LKKLLKLLKKLWKWAG